(19) United States
(12) Patent Application Publication
(10) Pub. No.: US 2016/0257644 A1
(43) Pub. Date: Sep. 8, 2016

(54) ACRYLONITRILE MANUFACTURE

(71) Applicant: INEOS EUROPE AG, Rolle (CH)

(72) Inventors: Timothy Robert McDonel, Elburn, IL (US); Jay Robert Couch, Naperville, IL (US); David Rudolph Wagner, Naperville, IL (US); Paul Trigg Wachtendorf, Victoria, TX (US)

(73) Assignee: INEOS EUROPE AG, Rolle (CH)

(21) Appl. No.: 15/060,759

(22) Filed: Mar. 4, 2016

(30) Foreign Application Priority Data
Mar. 6, 2015 (CN) 201510099369.7

(51) Int. Cl. C07C 253/26; B01D 17/02; B01J 19/24; C07C 253/24; C07C 253/34

(52) U.S. Cl. CPC C07C 253/26; C07C 253/24; C07C 253/34; B01J 19/24; B01D 17/0202; B01J 2219/24

(57) ABSTRACT

A method includes reacting, at a first pressure and in the presence of a catalyst, ammonia, oxygen, and a hydrocarbon selected from the group consisting of propane, propylene and isobutylene, and combinations thereof, to provide a reactor effluent stream that includes acrylonitrile. The method includes quenching the reactor effluent stream with a first aqueous stream to provide a quenched stream that includes acrylonitrile. The method includes compressing the quenched stream to provide an effluent compressor stream comprising acrylonitrile, and conveying, at a second pressure, the effluent compressor stream to an absorber. The method includes, in the absorber, absorbing acrylonitrile in a second aqueous stream to provide a rich water comprising acrylonitrile, wherein the absorbing is performed at a pressure greater than the first pressure.

### FIELD OF THE INVENTION

The invention relates to an improved process in the manufacture of acrylonitrile and methacrylonitrile. In particular, the invention is directed to an improved process using an effluent compressor.

### BACKGROUND

Various processes and systems for the manufacture of acrylonitrile and methacrylonitrile are known; see for example, U.S. Pat. No. 6,107,509. Typically, recovery and purification of acrylonitrile/methacrylonitrile produced by the direct reaction of a hydrocarbon selected from the group consisting of propane, propylene or isobutylene, ammonia and oxygen in the presence of a catalyst has been accomplished by transporting the reactor effluent containing acrylonitrile/methacrylonitrile to a first column (quench) where the reactor effluent is cooled with a first aqueous stream, transporting the cooled effluent containing acrylonitrile/methacrylonitrile into a second column (absorber) where the cooled effluent is contacted with a second aqueous stream to absorb the acrylonitrile/methacrylonitrile into the second aqueous stream, transporting the second aqueous stream containing the acrylonitrile/methacrylonitrile from the second column to a first distillation column (recovery column) for separation of the crude acrylonitrile/methacrylonitrile from the second aqueous stream, and transporting the separated crude acrylonitrile/methacrylonitrile to a second distillation column (heads column) to remove at least some impurities from the crude acrylonitrile/methacrylonitrile, and transporting the partially purified acrylonitrile/methacrylonitrile to a third distillation column (product column) to obtain product acrylonitrile/methacrylonitrile. U.S. Pat. Nos. 4,234,510; 3,885,928; 3,352,764; 3,198,750 and 3,044,966 are illustrative of typical recovery and purification processes for acrylonitrile and methacrylonitrile.

In a conventional process, the reactor pressure is constrained by the absorber off-gas pressure and the minimum necessary pressure drop between the reactor and the absorber. In a conventional process, this predetermined pressure in the reactor is about 8 psig and typically results in a conversion rate of about 80% of hydrocarbon feed to reactor product effluent comprising acrylonitrile. The reactor product effluent is then conveyed to a quench vessel. In the quench vessel, the reactor product effluent is quenched to produce quenched effluent comprising acrylonitrile product. The quenched effluent is conveyed to an absorber via a pipe. In the absorber, the quenched effluent is combined with refrigerated water to produce rich water comprising acrylonitrile, and off-gas from the absorber is burned in an absorber off-gas incinerator (AOGI) or absorber off-gas oxidizer (AOGO). The rich water comprising acrylonitrile that is generated in the absorber is then conveyed from the absorber to a recovery column for further processing. In a conventional process, the absorber is run at atmospheric pressure and refrigerated or cooled water is added to the absorber to mix with the quenched acrylonitrile product and generate the rich water comprising acrylonitrile.

While the manufacture of acrylonitrile/methacrylonitrile including the recovery and purification have been commercially practiced for years there are still areas in which improvement would have a substantial benefit. One of those areas for improvement would be increased reactor product conversion. Another improvement would be reducing the need for refrigerated or cooled water in the absorber.

### SUMMARY

An aspect of the disclosure is to provide a safe, effective and cost effective method and apparatus that overcomes or reduces the disadvantages of conventional processes.

A process includes reacting, at a first pressure and in the presence of a catalyst, ammonia, oxygen, and a hydrocarbon selected from the group consisting of propane, propylene, isobutane and isobutylene, and combinations thereof, to provide a reactor effluent stream that includes acrylonitrile. The process further includes quenching the reactor effluent stream with a first aqueous stream to provide a quenched stream that includes acrylonitrile and compressing the quenched stream to provide an effluent compressor stream that includes acrylonitrile. The process includes conveying, at a second pressure, the effluent compressor stream to an absorber and in the absorber, absorbing acrylonitrile in a second aqueous stream to provide a rich water that includes acrylonitrile, wherein the second pressure is greater than the first pressure.

A process includes reacting, at a first pressure and in the presence of a catalyst, ammonia, oxygen, and a hydrocarbon selected from the group consisting of propane, propylene, isobutane and isobutylene, and combinations thereof, to provide a pressurized reactor off-gas. The process further includes conveying the pressurized off-gas to an absorber and expanding a non-absorbed effluent from the absorber.

An apparatus includes a reactor configured to react, at a first pressure and in the presence of a catalyst, ammonia, oxygen, and a hydrocarbon selected from the group consisting of propane, propylene and isobutylene, and combinations thereof, to provide a reactor effluent stream comprising acrylonitrile; a quench vessel configured to quench the reactor effluent stream with a first aqueous stream to provide a quenched stream comprising acrylonitrile; an effluent compressor configured to compress the quenched stream to provide an effluent compressor stream comprising acrylonitrile at a second pressure; and an absorber configured to receive the effluent compressor stream and allow for absorbing of the acrylonitrile in a second aqueous stream to provide a rich water comprising acrylonitrile.

An ammoxidation process includes reacting ammonia, oxygen, and a hydrocarbon selected from the group consisting of propane, propylene, isobutane and isobutylene, and combinations thereof in the presence of a catalyst, at a pressure (absolute) of about 140 kPa or less and a velocity of about 0.5 to about 1.2 meters/second to provide a reactor effluent stream.

A process for absorbing a reactor effluent stream that includes acrylonitrile, includes quenching the reactor effluent stream with a first aqueous stream to provide a quenched stream that includes acrylonitrile; compressing the quenched stream to provide an effluent compressor stream that includes acrylonitrile; conveying the effluent compressor stream to an absorber at a pressure (absolute) of about 300 kPa to about 500 kPa; and in the absorber, absorbing acrylonitrile in a second aqueous stream to provide a rich water that includes acrylonitrile.

In another aspect, a process for absorbing a reactor effluent stream that includes acrylonitrile, the process includes quenching the reactor effluent stream with a first aqueous stream to provide a quenched stream that includes acrylonitrile; compressing the quenched stream to provide an effluent compressor stream that includes acrylonitrile; conveying the effluent compressor stream to an absorber; and in the absorber, absorbing acrylonitrile in a second aqueous stream having a temperature of about 4° C. to about 45° C. to provide a rich water that includes acrylonitrile.

In another aspect, an ammoxidation system includes a turbine effective for driving a single drive line that includes an air compressor and at least one effluent compressor.

In another aspect, an ammoxidation process includes reacting ammonia, oxygen, and a hydrocarbon selected from the group consisting of propane, propylene, isobutane and isobutylene, and combinations thereof in the presence of a catalyst, at a pressure of about 100 kPa (absolute) or less and a velocity of about 0.5 to about 1.2 meters/second to provide a reactor effluent stream.

In a related aspect, an ammoxidation apparatus includes a reactor configured to react, at a first pressure of about 100 kPa (absolute) or less and in the presence of a catalyst, ammonia, oxygen, and a hydrocarbon selected from the group consisting of propane, propylene and isobutylene, and combinations thereof, to provide a reactor effluent stream comprising acrylonitrile.

In another aspect, an ammoxidation system includes a turbine for driving a single drive operatively coupled to at least one air compressor and at least one effluent compressor, the air compressor configured to provide air to an ammoxidation reactor, the effluent compressor configured to provide an effluent compressor stream to an absorber, and the ammoxidation reactor and absorber configured to allow for independent pressure control.

The above and other aspects, features and advantages of the present disclosure will be apparent from the following detailed description of the illustrated embodiments thereof which are to be read in connection with the accompanying drawings.

### BRIEF DESCRIPTION OF THE DRAWINGS

A more complete understanding of the exemplary embodiments of the present invention and the advantages thereof may be acquired by referring to the following description in consideration of the accompanying drawings in which like reference numbers indicate like features and wherein:

FIG. 1 is a schematic flow diagram of an embodiment in accordance with aspects of the disclosure as applied to the manufacture of acrylonitrile.

FIG. 2 is a schematic flow diagram of another embodiment in accordance with aspects of the disclosure as applied to the manufacture of acrylonitrile.

FIG. 3 is a schematic flow diagram illustrating an aspect that includes more than one reactor, quench column and effluent compressor.

FIG. 4 is a schematic flow diagram illustrating a single line drive.

### DETAILED DESCRIPTION

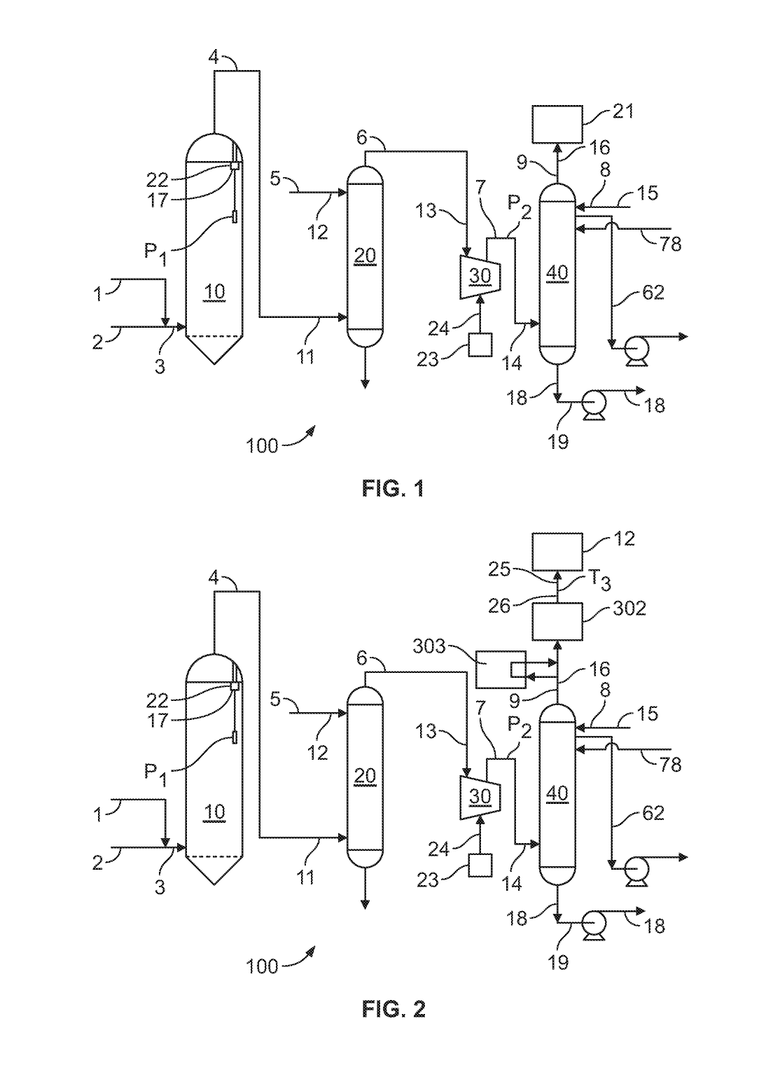

FIG. 1 is a schematic flow diagram of an embodiment in accordance with aspects of the disclosure as applied to the manufacture of acrylonitrile. Referring to the figure, an apparatus 100 comprises reactor 10, quench vessel 20, effluent compressor 30, and absorber 40 Ammonia in stream 1 and hydrocarbon (HC) feed in stream 2 may be fed as combined stream 3 to reactor 10. HC feed stream 2 may comprise a hydrocarbon selected from the group consisting of propane, propylene and isobutylene, and combinations thereof. A catalyst (not shown in FIG. 1) may be present in reactor 10. Oxygen containing gas may be fed to reactor 10. For example, air may be compressed by an air compressor (not shown in FIG. 1) and fed to reactor 10.

Acrylonitrile is produced in reactor 10 from the reaction of the hydrocarbon, ammonia, and oxygen in the presence of a catalyst in reactor 10. Reactor 10 may be run at reactor or first pressure P1, wherein the first pressure may be characterized as the pressure at inlet 17, such as a first-stage inlet of cyclone 22. In accordance with the disclosure, cyclone 22 may be the first cyclone of a multi-stage cyclone system that may be configured to convey a stream comprising acrylonitrile to a plenum (not shown in FIG. 1). The stream comprising acrylonitrile may exit the plenum and out of a top portion of reactor 10 as reactor effluent stream 4. In an aspect, cyclone 22 may be configured to separate catalyst that may be entrained in the stream comprising acrylonitrile that enters inlet 17, and return the separated catalyst back to the catalyst bed in reactor 10 through a catalyst return dip leg (not shown in FIG. 1). Reactor effluent stream 4 comprising acrylonitrile produced in reactor 10 may be conveyed through line 11 to quench vessel 20. In this aspect, the first pressure is about 140 kPa or less, in another aspect about 135 kPa or less, in another aspect about 130 kPa or less, in another aspect about 125 kPa or less, in another aspect, about 101 kPa to about 140 kPa, in another aspect, about 110 kPa to about 1400 kPa, in another aspect, about 125 kPa to about 145 kPa, in another aspect, about 120 kPa to about 140 kPa, in another aspect, about 130 kPa to about 140 kPa, in another aspect, about 125 kPa to about 140 kPa, in another aspect, about 125 kPa to about 135 kPa, in another aspect, about 120 kPa to about 137 kPa, and in another aspect, about 115 kPa to about 125 kPa.

In quench vessel 20, reactor effluent stream 4 may be cooled by contact with quench aqueous stream 5 entering quench vessel 20 via line 12. Quench aqueous stream 5 may comprise an acid in addition to water. The cooled reactor effluent comprising acrylonitrile (including co-products such as acetonitrile, hydrogen cyanide and impurities) may then be conveyed as quenched stream 6 to effluent compressor 30 via line 13.

Quenched stream 6 may be compressed by effluent compressor 30, and exit effluent compressor 30 as compressor effluent stream 7. Compressor effluent stream 7 may have a second or compressed pressure P2. Compressor effluent stream 7 may be conveyed to a lower portion of absorber 40 via line 14. In absorber 40, acrylonitrile may be absorbed in a second or absorber aqueous stream 8 that enters an upper portion of absorber 40 via line 15. The aqueous stream or rich water stream 18 that include acrylonitrile and other co-products may then be transported from absorber 40 via line 19 a recovery column (not shown in FIG. 1) for further product purification.

The non-absorbed effluent 9 exits from the top of absorber column 40 through pipe 16. Non-absorbed effluent 9 may comprise off-gases, which can be burned in absorber off-gas incinerator (AOGI) or absorber off-gas oxidizer (AOGO).

In an aspect, effluent compressor 30 functions by pulling quenched stream 6 through line 13. Effluent compressor 30 may compress quenched stream 6 so that it exits effluent compressor 30 as compressed effluent compressor stream 7 that has a higher pressure (second pressure) than the reactor pressure (first pressure). In an aspect, the pressure in line 14 of compressed effluent compressor stream 7 is about 2 to about 11.5 times greater than the operation pressure of reactor 10, in another aspect, about 2 to about 12.5 times, in another aspect, about 2.5 to about 10, in another aspect, about 2.5 to about 8, in another aspect, about 2.5 to about 5, in another aspect, about 2.5 to about 4, in another aspect, about 2.5 to about 3.2, in another aspect, about 2 to about 3.5, in another aspect, about 2 to about 3, in another aspect, about 3 to about 11.25, in another aspect, about 5 to about 11.25, and in another aspect, about 7 to about 11.25 (all based on an absolute comparison). In an aspect, the second pressure (absolute) is about 300 to about 500 kPa, in another aspect, about 340 kPa to about 415 kPa, in another aspect, about 350 kPa to about 400 kPa, in another aspect, about 250 kPa to about 500 kPa, in another aspect, about 200 kPa to about 400 kPa, in another aspect, about 250 kPa to about 350 kPa, in another aspect, about 300 kPa to about 450 kPa, and in another aspect, about 360 kPa to about 380 kPa.

In an aspect, the second pressure is such that the absorber may be operated with a flow rate of aqueous stream 8 of about 15 to about 20 kg/kg of acrylonitrile final product produced when aqueous stream 8 is uncooled or unrefrigerated and/or is 4 to about 45° C. and wherein the absorber rich water stream contains about 5 weight percent or more organics, in another aspect, about 6 weight percent or more organics, and in another aspect, about 7 weight percent or more organics. In another aspect, the flow rate of aqueous stream 8 may be about 15 to about 19 kg/kg of acrylonitrile, in another aspect, about 15 to about 18 kg/kg of acrylonitrile, and in another aspect, about 16 to about 18 kg/kg of acrylonitrile. In another aspect, the uncooled or unrefrigerated aqueous stream is about 20 to about 45° C., in another aspect, about 25 to about 40° C., in another aspect, about 25 to about 35° C., and in another aspect, about 25 to about 30° C.

A cooling system (not shown in FIG. 1) may be located at or downstream of compressor 30, wherein the cooling system is configured to cool compressed effluent compressor stream 7 to a predetermined temperature, e.g., about 105° F. (about 40.5° C.) prior to entering absorber 40.

In an aspect, absorber 40 may include forty to sixty (40-60) trays. In an aspect, absorber 40 may include fifty (50) trays. Compressed effluent compressor stream 7 may enter absorber 40 below the bottom tray of the absorber. In an aspect, absorber 40 may be operated with variable flow rates of refrigerated water in second aqueous stream 8, including zero amount of refrigerated water.

In an aspect, absorber 40 may be operated at pressure that is higher than the pressure in an absorber in a conventional process. By operating absorber 40 at this higher pressure, the absorber may be operated more efficiently than an absorber in a conventional process. Due to the higher absorber efficiency achieved in the process of the present disclosure, the same recovery of acrylonitrile in rich water stream 18 may be achieved as in a conventional process, but less water is required to absorb acrylonitrile in the absorber. In this aspect, rich water refers to water having about 5 weight percent or more organics, in another aspect, about 6 weight percent or more organics, and in another aspect, about 7 weight percent or more organics. In an aspect, the water used to absorb acrylonitrile in the absorber may be process or municipal water (e.g., having a temperature of about 4-45° C.). In this aspect, process or municipal water is more than about 95 weight percent water, in another aspect, about 97 weight percent or more water, in another aspect, about 99 weight percent or more water, and in another aspect, about 99.9 weight percent or more water. In an aspect, the temperature of second aqueous stream 8 may be in the range of about 4 to about 45° C., in another aspect, about 10 to about 43° C., and in another aspect, about 27 to about 32° C.

In an aspect, aqueous stream 8 may be devoid of cooled or refrigerated water. In an aspect, aqueous stream 8 may have a higher temperature than the temperature required in an aqueous stream in a conventional process. In an aspect, aqueous stream 8 may comprise refrigerated water, and when aqueous stream 8 comprises refrigerated water, the flow rate of aqueous water stream 8 may be less than the flow rate required in an aqueous stream in a conventional process. In this aspect, the first aqueous stream has a temperature of about 20° C. to about 50° C., in another aspect, about 25° C. to about 45° C., and in another aspect, about 30° C. to about 40° C. The first aqueous stream may be provided to the absorber at a rate of about 25 kg to about 35 kg first aqueous stream per kg of acrylonitrile produced, and in another aspect, about 27 kg to about 33 kg first aqueous stream per kg of acrylonitrile produced.

In an aspect, reactor 10 may be operated under a pressure that is lower than the pressure required in a conventional process. In a conventional process, which does not have an effluent compressor, reactor 10 typically needs to be run at a pressure of about 8 psig to achieve a conversion rate of, for example, 80% or more of hydrocarbon feed to effluent product comprising acrylonitrile. In an aspect of the present disclosure, the process includes operating reactor 10 at pressure that is about 35 to about 50% (absolute basis) lower than in a conventional process. In an aspect of the present disclosure, the process comprises operating reactor 10 at a pressure of about 4-5 psig. It has been found that by lowering the operating pressure of reactor 10 in accordance with the present disclosure, a conversion rate of at least about 70% or more, in another aspect, about 75% or more, in another aspect, about 81% or more, and in another aspect, about 82% or more of hydrocarbon feed to acrylonitrile can be achieved.

The fluidized bed reactor is at the heart of an acrylonitrile plant. Failure to correctly design a new reactor could at a minimum significantly affect the efficiency, reliability or production capacity of an entire acrylonitrile plant and in the extreme lead to an extended shut-down of production whilst reactor modifications or change-out could be implemented. The operation of a fluidized bed is highly sensitive to the specific operating conditions selected and the industry is highly cautious in changing operating conditions and/or the design of the reactor or its internals. As the operating window (eg pressure and fluidization velocity) or fluidized bed characteristics change (eg. reactor diameter, internals, bed height, ratio of bed pressure drop to grid pressure drop) and catalyst characteristics change (particle size, particle size distribution, fines content, attrition characteristics), so too can the critical circulation patterns in the fluidized bed.

One of the most sensitive parameters which could affect fluidization performance is the scale-up of the reactor diameter. In this aspect, a reactor may have an internal diameter of about 5 to about 15 meters, in one aspect, about 7 to about 12 meters, in another aspect, about 8 to about 11 meters, and in another aspect, about 9 to about 11 meters. Reactor diameter is also one of the parameters that leads to most scale-up caution since there are limited mitigation options available, absent reactor change-out, to correct a scale-up of diameter that has gone too far. Through significant experimentation and optimization, it has now been found that, when using a catalyst with an average particle diameter between about 10 and 100μ, with a particle size distribution where about 0 to 30 weight percent is greater than about 90μ, and about 30 to 50 weight percent is less than 45μ, a reactor internal diameter of greater than about 9 m up to about 11 m can be combined with suitable operating conditions and reactor internals to achieve acceptable fluidization conditions for the production of acrylonitrile and methacrylonitrile whilst operating at reactor pressures of about 140 kPa or less, in another aspect about 135 kPa or less, in another aspect about 130 kPa or less, in another aspect about 125 kPa or less, in another aspect, about 101 kPa to about 140 kPa, in another aspect, about 110 kPa to about 140 kPa, in another aspect, about 125 kPa to about 145 kPa, in another aspect, about 120 kPa to about 140 kPa, in another aspect, about 130 kPa to about 140 kPa, in another aspect, about 125 kPa to about 140 kPa, in another aspect, about 125 kPa to about 135 kPa, in another aspect, about 120 kPa to about 137 kPa, and in another aspect, about 115 kPa to about 125 kPa.

As the reactor pressure is decreased so it is necessary to increase reactor diameter and/or reactor velocity in order achieve a given production rate of acrylonitrile. It has furthermore been found that at these reduced reactor pressures, optionally at larger diameters, it is also possible to operate a relatively high bed height to bed diameter ratios, thus maximizing the catalyst inventory whilst minimizing the increase in the diameter. In one aspect, the air grid design provides about a 30 to 40% (minimum) of catalyst bed pressure drop at reactor turndown. It has also been determined that so long as the catalyst is within the above range of particle characteristics and preferably has an attrition loss of between about 1 and 4%, the fluidization velocity (based on effluent volumetric flow and reactor cross-sectional area (“CSA”) excluding cooling coils and dip legs area) can be operated at up to 1.2 m/s, preferably between 0.55 and 1, for reactors having a 9 to 11 m internal diameter. Attrition loss may be determined using known methods, such as for example, Hartge et al., The 13^{th} International Conference on Fluidization—New Paradigm in Fluidization Engineering, Art. 33 (2010), methods based on ASTM D4058 and ASTM D5757, and U.S. Pat. No. 8,455,388 which are all incorporated herein in their entirety by reference. In a related aspect, total catalyst loss from the reactor may be about 0.35 to about 0.45 kg/metric ton of acrylonitrile produced.

Even at up to the indicated velocities it has been found possible to operate with acceptable catalyst loses while operating the reactor with a top pressure of about 0.25 to about 0.45 kg/cm² and/or cyclones with a pressure drop of 15 kPa or less, and a fines disengagement height above the top of the fluidized bed of about 5.5 to about 7.5 m. Thus when utilizing a reactor internal diameter of about 9 to about 11 m according to this invention, using a catalyst with an average particle diameter between about 10 and 100μ, with a particle size distribution where about 0 to about 30 weight percent is greater than about 90μ, and about 30 to about 50 weight percent is less than 45μ, a ratio of reactor diameter to a reactor cylindrical height (tangent to tangent) of about 0.45 to about 0.6 has been found to be effective when operating with a fluidization velocity (based on effluent volumetric flow and reactor cross-sectional area excluding cooling coils and dip leg area) between about 0.4 m and 1.05 m/s preferably between about 0.55 and 0.85 m/s. This therefore leads to potential for increased production capacity per unit reactor volume (tangent to tangent) of between 0.005 and 0.015 metric tons per hour per cubic meter of reactor volume, in another aspect, about 0.0075 to about 0.0125, and in another aspect, about 0.009 to about 0.01 metric tons per hour per cubic meter of reactor volume.

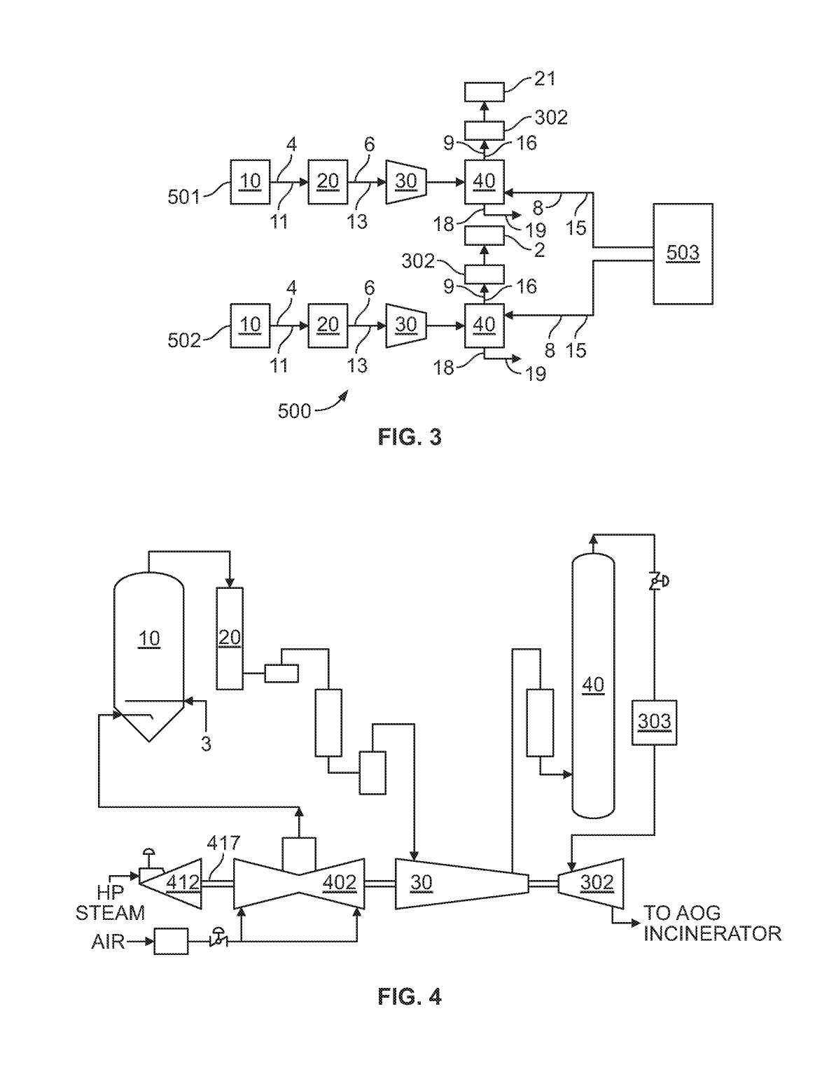

It is desirable to ensure that reactor efficiency (including in terms of reagent conversion and catalyst losses) is optimized whilst increasing the specific capacity of the reactor. The design of the cyclone is critical to the operating pressure of the reactor, the catalyst losses (including caused by attrition) and the required height of the reactor (tangent to tangent). It has been found that the above satisfactory reactor operating window can be achieved with ratio of first stage cyclone inlet velocities to reactor effluent velocities of about 20 to about 30 and/or ratio of a height of a first stage cyclone is about 4% to about 7% of a reactor height (tangent to tangent). As shown in FIG. 3, the cyclone height is determined from a distance from a top 101 of the cyclone to a distal section 107 of the cyclone.

In an aspect, reactor 10 may be configured to have a greater throughput capacity for a predetermined catalyst than a conventional reactor having the same predetermined catalyst and predetermined reactor height. In an aspect, a method is provided for increasing reactor throughput capacity for a predetermined catalyst and predetermined reactor height. The method includes increasing reactor diameter while maintaining a predetermined top pressure. The method may comprise maintaining a predetermined reactor design velocity.

In an aspect, a process includes operating or reacting in a reactor a hydrocarbon, wherein the reactor has a predetermined reactor internal diameter of more than about 40% to about 60% a cylindrical height of the reactor (tangent to tangent), and in another aspect, about 45% to about 55%. This is in contrast to a conventional process that includes operating a reactor having a reactor diameter that is about 40% of the reactor height. In a related aspect, reactor height (tangent to tangent) may be about 10 to about 25 meters, in another aspect, about 10 to about 20 meters, in another aspect, about 12 to about 18 meters, and in another aspect, about 14 to about 16 meters.

In an aspect, the process includes operating or reacting in a reactor a hydrocarbon, wherein the reactor has a fluidized bed height that is about 40% to about 60% of the reactor cylindrical height (tangent to tangent), in another aspect, about 42% to about 50%, in another aspect, about 45% to about 55%, and in another aspect, about 44% to about 47%. This is in contrast to a conventional process that includes operating a reactor having a fluidized bed height that is about 25% of the reactor height (tangent to tangent) and thus, a greater disengagement height.

In an aspect, the process includes operating or reacting in a reactor a hydrocarbon, wherein the reactor has a fluidized bed height that is about 70% to about 110% of the reactor diameter, in another aspect, about 70% to about 100%, in another aspect, about 75% to about 90%, in another aspect, about 80% to about 90%, in another aspect, about 85% to about 95%, and in another aspect, about 85% to about 90%. This is in contrast to a conventional process that includes operating a reactor having a fluidized bed height that is about 65% of the reactor diameter.

In an aspect, the process includes operating or reacting in a reactor a hydrocarbon, wherein the reactor has a top pressure in the range of about 0.25 to about 0.45 kg/cm², in another aspect, about 0.3 to about 0.5 kg/cm², in another aspect, about 0.2 to about 0.4 kg/cm², and in another aspect, about 0.2 to about 0.5 kg/cm². A reactor top pressure in this range provides the benefit of improved catalyst performance over a reactor top pressure that is higher than this range. In an aspect, the method includes operating the reactor in the range of about 0.4 to about 0.45 kg/cm².

In an aspect, the method includes operating or reacting in a reactor a hydrocarbon, wherein the effluent volumetric flow has a linear velocity of about 0.5 to about 1.2 m/sec (based on effluent volumetric flow and reactor cross-sectional area (“CSA”) excluding cooling coils and dip legs area, i.e., ˜90% of open CSA). It has been found that it is possible to design and operate the reactor system using this velocity whilst also achieving good fluidization/catalyst performance and reasonable catalyst entrainment/catalyst losses from cyclones, such that velocities may be maintained in about this range to the extent possible as reactor capacity is increased. In an embodiment, the reactor may be operated with a velocity of up to about 0.75 m/sec to about 0.95 m/sec (based on 90% CSA and effluent gas), and maintain a top pressure of about 0.25 to about 0.5 kg/cm², and in another aspect, about 0.2 to about 0.45 kg/cm². In one aspect, a ratio of cyclone inlet velocity in meters/second to a reactor effluent velocity in meters/second is 20 or greater, in another aspect, about 20 to about 30, in another aspect, about 22 to about 25, in another aspect, about 23 to about 26, and in another aspect, about 27 to about 29.

As the fluidization velocity is increased so too does the potential for attrition of the catalyst increase. Increased velocity also results in a greater fines disengagement height above the fluidized bed. The resultant increase in fines can therefore also increase the solids loading on the cyclones.

In an aspect, it has been found that by operating a reactor or reacting in a reactor a hydrocarbon, wherein the reactor has a predetermined reactor diameter having a length that is in the range of about 45% to about 60% a length of the reactor height, a length of fluidized bed height that is about 80% to about 95% the length of the reactor diameter, a pressure in the range of about 0.3 to about 0.5 kg/cm², and a reactor velocity (based on 90% CSA and effluent gas) of about 0.6 to about 0.65 m/sec, the process may produce up to about 100% or more acrylonitrile product than a method wherein reactor is operated wherein the reactor diameter is about 40% of the reactor height, the fluidized bed height is about 25% of the reactor height, and the fluidized bed height is about 65% of the reactor diameter.

In an aspect, where the reactor diameter is at least 8 m internal diameter and uses the optimized combination of features above, the apparatus and method provides a reactor capacity that is about 12.5 metric tons/hr or 100 ktpa per reactor based on 8000 operating hours per year. Where the reactor diameter is 10.5 m the single reactor capacity can be between 15 and 20 metric tons/hr.

Determination of Fluidized Bed Height for Purposes of this Application

The reactor needs to be equipped with at least 3 nozzles for measuring fluidized bed differential pressures as listed below:

1) The 1st of these nozzles is located near the bottom of the fluidized bed (above the air distributor). In this aspect, the nozzles may be about 0.1 to about 0.7 meters above the air distributor, and in another aspect, about 0.2 to about 0.4 meters.
2) The 2nd nozzle is typically located about 2 meters above the 1st nozzle (still within the fluidized bed). The exact distance must be known for the calculations.
3) The 3rd nozzle is located at the top of the reactor (above the fluidized bed).

By measuring the pressure difference between the 1st and 2nd nozzles and also measuring the pressure difference between the 1st and 3rd nozzles, the bed height may be calculated as follows:

Bed Height=(distance between the 1st and 2nd nozzles)×(1st-3rd differential pressure)/(1st-2nd differential pressure)

Note that the fluidized bed density is assumed to be approximately constant in the above formula.

The units for the two pressure measurements need to be the same for each but can be any typical unit of pressure (for example lbs/in², inches of water column, or millimeters of water column).

The units for the distance between the taps can be any typical unit of distance (for example feet or meters). The bed height will be in the same units chosen.

Differential pressures are preferably measured with two differential pressure transmitters—one for the 1st-2nd nozzle differential pressure measurement and one for the 1st-3rd nozzle differential pressure measurement. The nozzles are typically purged with flowing air to keep them clear. In this aspect, air velocity for nozzle purge is about 2 to about 8 m/sec.

In an aspect, apparatus 100 may comprise a water spray system 23 (as shown in FIG. 1 and FIG. 2) that is configured to spray water stream 24 to at least one surface of effluent compressor 30 to reduce fouling on that surface. In an aspect, a variable speed turbine may be used with the effluent compressor 30.

In an aspect, when reactor 10 is operated with a top pressure of about 5 psig, the cooled effluent gas must be compressed before further processing can take place. In an aspect, gas leaves a compressor suction separator and flows to a first section of effluent compressor 30. Demineralized water may be spray-injected into the compressor suction line and also into diffuser passages. This water injection may be configured to maintain a water film on rotating and stationary surfaces so that deposits will not accumulate. The water injection may be configured to minimize the gas (and therefore minimize the rate of polymer formation) throughout the effluent compressor. The gas temperature may be lowered by vaporization of some of the water spray. The net benefit may be an acceptable service factor for the effluent compressor.

Gas from the first section of the effluent compressor may be passed through an effluent compressor intercooler. The cooled gas may flow to a compressor inter-stage separator, in which condensate is removed. Gas from the separator may be conveyed to a second section of the effluent compressor, wherein water sprays may be used in the same manner as in the first section of effluent compressor 30. From the second section, the effluent gas may be cooled in an effluent compressor aftercooler or exchanger. A mixture of gas and condensate may leave the aftercooler or exchanger and enter absorber 40 below the bottom tray of absorber 40.

Process condensate may be removed from the inter-stage separator and returned by pressure-difference to the suction separator, where it mixes with condensate from a secondary effluent cooler upstream of the effluent compressor. The combined process condensate may be recycled to the process-side inlets of the secondary effluent cooler and the compressor inter-stage cooler. Net condensate may be sent to the inlet of an aftercooler. The condensate may be sprayed over the tube sheets of these exchangers to provide a wash liquid to aid in keeping the insides of the exchanger tubes clean.

In an aspect, effluent compressor 30 may have casing and wheels configured to allow the injection of demineralized make-up water into the suction and wheel passages, in a predetermined amount to maintain a water film throughout the compressor. This wash water may be provided and controlled by a controller. Provision may be made for addition of inhibitor to the wash water.

In an aspect, effluent compressor 30 may be sized to handle reactor effluent gas after quenching and cooling to about 105° F. (40.5° C.). The gas rate and composition may be derived from predetermined yields and rates. A portion of the absorber off gas may be used as stripping gas for the quench bottoms stripper. This stripping gas may be returned to absorber 40 via effluent compressor 30, and the effluent compressor 30 may be sized for this incremental flow.

In an aspect, the effluent compressor frame size may be configured to allow for about 5% overcapacity. In another aspect, additional overcapacity up to about 35%, in another aspect, about 25%, in another aspect, about 15%, and in another aspect, about 10% total may be provided if within the same frame size. For each of the two sections, the maximum discharge temperature may be 200 degrees F. (93.3° C.), as maintained by water sprays.

In an aspect, when compressed effluent compressor stream 7 enters absorber 40, the process condensate phase may be conveyed to the absorber sump or utilized in other processes, while gas flows upwards through the absorber trays (which may be valve-type trays) against a descending stream of absorption water or second aqueous stream 8. Lean water for absorption may be withdrawn from the recovery column, cooled, and sent to the top tray of the absorber. In an aspect, no refrigeration is supplied to the lean water, and none is provided for any other portion of the absorber.

The gas leaving the top of the absorber is practically free of acrylonitrile and other organics, but may contain carbon monoxide and unconverted hydrocarbons such as propane. Environmental requirements may make it necessary to destroy these compounds before discharging of the off-gas to the atmosphere. Destruction of these compounds may be accomplished with an incinerator or oxidizer system, such as AOGO 21.

The absorber bottoms or rich water contains the recovered acrylonitrile and other organics, and this rich water stream may be sent to the acrylonitrile recovery column.

As shown in FIG. 3, apparatus 300 comprises the same features of apparatus 100, and further includes expander 302. Expander 302 may be configured to expand or reduce the pressure of non-absorbed effluent 9 from absorber 40 to a lower pressure. In an aspect, expander 302 may be configured to reduce the pressure of non-absorbed effluent 9 by a factor of about 17.5 to 22.5. In an aspect, expander 302 may be configured to reduce the pressure of non-absorbed effluent 9, which may be the same as the pressure in the absorber. For example, expander 302 may be configured to reduce the pressure of about 35-45 psig of non-absorbed effluent 9 to provide expanded non-absorbed effluent 25 having a lower pressure of about 2 psig or less. In this aspect, the expanding results in a reduction in pressure of the non-absorbed effluent from the absorber from a pressure (absolute) of about 300 kPa to about 500 kPa to a pressure (absolute) of about 115 kPa or less. Expanded non-absorbed effluent 25 may be conveyed from expander 302 to AOGO 21 via line 26.

Apparatus 300 may include pre-heater 303. Pre-heater 303 may be configured to pre-heat non-absorbed effluent 9 from a temperature of about 25° C. to about 40° C. to a temperature of about 350° C. or more, and in another aspect about 37.7° C. to a temperature of about 371.1° C. By pre-heating non-absorbed effluent 9 before it enters expander 302, condensing in expander 302 may be avoided or reduced. In an aspect, as non-absorbed effluent 9 expands in expander 302, its temperature is lowered is lowered from a temperature of about 300° C. to about 400° C. to a temperature of about 200° C. to about 260° C.

In an aspect, fuel gas required to burn expanded non-absorbed effluent 25 in absorber off-gas incinerator (AOGI) or absorber off-gas oxidizer (AOGO) 21 may be less than the fuel gas required to burn non-absorbed effluent 9, which has not been expanded. It has been found that by increasing the expander pressure drop in expander 302, less fuel gas may be required to burn expanded non-absorbed effluent 25 in AOGO 21. For example, it has been found that by increasing the pressure drop in expander 302, temperature T3 may be about 500° F. (about 260° C.) instead of about 400° F. (about 204.4° C.). When the expanded non-absorbed effluent 25 has a temperature T3 of about 500° F. (about 260° C.) as opposed to a temperature of about 400° F. (about 204.4° C.), less fuel gas is required to burn expanded non-absorbed effluent 25 in AOGO 21, and less water is needed to absorb acrylonitrile in absorber 40 to produce rich water stream 18. It has been discovered that it may be more cost-effective to deliberately lose a small amount of acrylonitrile product to reduce fuel gas needed for burning in AOGO 21 in certain applications.

As shown in FIG. 3, apparatus 500 may include a first train 501 and a second train 502. Each train may be similar to or the same as apparatus 100 or 300 previously described. As shown in FIG. 3, each train may include its own reactor 10, quench vessel 20, effluent compressor 30 and absorber 40, and the trains may be operated in parallel. In an aspect, each train may include its own AOGO 21. In an aspect, each absorber of each train may be configured to receive its own second or absorber aqueous stream 8 that is supplied in a line 15 that is separate from the other train. Each line 15 of each train may receive an absorber aqueous stream 8, wherein stream 8 was used in operation or generated in operation of acrylonitrile recovery column 503. Rich water streams 18 from each absorber of each train may be conveyed to the acrylonitrile recovery column 503. These rich water streams may be combined before further processing.

In one aspect, an ammoxidation system includes a turbine effective for driving a single drive line that includes an air compressor and at least one effluent compressor. The turbine may be selected from the group consisting of a steam turbine, a gas turbine, an electric turbine, and a variable speed electric turbine. As shown in FIG. 4, high pressure steam is provided to a steam turbine 412. In this aspect, steam provided to the steam turbine 412 has a pressure of about 600 psig or more, and in another aspect, about 600 to about 700 psig. The steam turbine 412 is effective for driving a single drive line 417 that includes one or more air compressors 402, one or more effluent compressors 30, and at least one expander 302. The air compressor 402 is configured to provide air to the reactor 10, and the effluent compressor 30 is configured to provide an effluent stream to the absorber 40.

In another aspect, the reactor 10 and absorber 40 may each include valves (not shown). The valves are configured to allow independent control of reactor 10 and absorber 40. For example, at start-up, the valve on the reactor 10 may be vented to atmosphere to prevent formation of a vacuum in the reactor 10.

In an aspect, the method and apparatus of the present disclosure provides more flexibility in operation than conventional methods and apparatuses. For example, the method and apparatus of the present disclosure provides more flexibility in turndown or using lower rates when less production of acrylonitrile is needed than in conventional methods and apparatuses.

An effluent compressor typically is less expensive than chiller equipment that is required to provide refrigerated water to an absorber in a conventional process. For this reason, the apparatus and method present disclosure may have a lower capital expenditure than for conventional apparatus and method.

In an aspect, it has been found that the above method may achieve a higher reactor product conversion than in a conventional process that does not include compressing a quenched stream and absorbing at a third pressure that is greater than the first pressure. In an aspect, it has been found that by operating the absorber at a higher pressure than the pressure in an absorber in a conventional process, rich water comprising acrylonitrile may be generated in the absorber with either less refrigerated water and/or water that is warmer than the refrigerated water used as the absorbing aqueous stream in conventional processes.

In another aspect, an ammoxidation process includes reacting ammonia, oxygen, and a hydrocarbon selected from the group consisting of propane, propylene, isobutane and isobutylene, and combinations thereof in the presence of a catalyst, at a pressure of about 100 kPa (absolute) or less and a velocity of about 0.5 to about 1.2 meters/second to provide a reactor effluent stream. The reacting may be conducted at a pressure of about 5 kPa (absolute) to about 100 kPa (absolute), in another aspect, about 10 kPa (absolute) to about 90 kPa (absolute), in another aspect, about 20 kPa (absolute) to about 80 kPa (absolute), in another aspect, about 30 kPa (absolute) to about 70 kPa (absolute), and in another aspect, about 40 kPa (absolute) to about 60 kPa (absolute). It has been found that by lowering the operating pressure of reactor 10 as indicated, a conversion rate of at least about 70% or more, in another aspect, about 75% or more, in another aspect, about 81% or more, and in another aspect, about 82% or more of hydrocarbon feed to effluent product comprising acrylonitrile can be achieved.

While in the foregoing specification this disclosure has been described in relation to certain preferred embodiments thereof, and many details have been set forth for purpose of illustration, it will be apparent to those skilled in the art that the disclosure is susceptible to additional embodiments and that certain of the details described herein can be varied considerably without departing from the basic principles of the disclosure. It should be understood that the features of the disclosure are susceptible to modification, alteration, changes or substitution without departing from the spirit and scope of the disclosure or from the scope of the claims. For example, the dimensions, number, size and shape of the various components may be altered to fit specific applications. Accordingly, the specific embodiments illustrated and described herein are for illustrative purposes only.

We claim:

1. An ammoxidation process comprising:
reacting ammonia, oxygen, and a hydrocarbon selected from the group consisting of propane, propylene, isobutane and isobutylene, and combinations thereof in the presence of a catalyst, at a pressure of about 100 kPa (absolute) or less and a velocity of about 0.5 to about 1.2 meters/second to provide a reactor effluent stream.

2. The ammoxidation process of claim 1, wherein the reactor has an internal diameter of about 5 to about 15 meters.

3. The ammoxidation process of claim 1, wherein the reactor has an internal diameter of about 9 to about 12 meters.

4. The ammoxidation process of claim 1, wherein the reactor has a height (tangent to tangent) of about 10 to about 25 meters.

5. The ammoxidation process of claim 1 wherein the reacting is conducted at a pressure of about 5 kPa (absolute) to about 100 kPa (absolute).

6. The ammoxidation process of claim 1, wherein the velocity is measured at an inlet of the reactor and pressure is measured at an inlet of a cyclone.

7. The ammoxidation process of claim 1, wherein the process is effective for providing a conversion rate of hydrocarbon feed to acrylonitrile of about 70% or more.

8. The ammoxidation process of claim 1, further comprising quenching the reactor effluent stream with a first aqueous stream to provide a quenched stream that includes acrylonitrile;
compressing the quenched stream to provide an effluent compressor stream that includes acrylonitrile;
conveying, at a pressure of about more than 300 kPa (absolute) to about 500 kPa (absolute), the effluent compressor stream to an absorber; and
in the absorber, absorbing acrylonitrile in a second aqueous stream to provide a rich water that includes acrylonitrile.

9. The ammoxidation process of claim 8, further comprising expanding non-absorbed effluent from the absorber to reduce the pressure of the non-absorbed effluent.

10. The ammoxidation process of claim 9, wherein the expanding results in a reduction in pressure of the non-absorbed effluent from the absorber to a pressure of about 150 kPa (absolute) or less.

11. The ammoxidation process of claim 9, further comprising pre-heating the non-absorbed effluent from the absorber prior to the step of expanding.

12. The ammoxidation process of claim 9, wherein the pre-heating raises the temperature of the non-absorbed effluent from a temperature of about 25° C. to about 40° C. to a temperature of about 350° C. or more.

13. The ammoxidation process of claim 9, wherein during the step of expanding, the temperature of the non-absorbed effluent is lowered from a temperature of about 300° C. to about 400° C. to a temperature of about 200° C. to about 260° C.

14. An ammoxidation apparatus comprising:
a reactor configured to react, at a first pressure of about 100 kPa (absolute) or less and in the presence of a catalyst, ammonia, oxygen, and a hydrocarbon selected from the group consisting of propane, propylene and isobutylene, and combinations thereof, to provide a reactor effluent stream comprising acrylonitrile.

15. The ammoxidation apparatus of claim 14 wherein the reactor is configured to react at a pressure of about 5 kPa (absolute) to about 100 kPa (absolute).

16. The ammoxidation apparatus of claim 14 further comprising
a quench vessel configured to quench the reactor effluent stream with a first aqueous stream to provide a quenched stream comprising acrylonitrile;
an effluent compressor configured to compress the quenched stream to provide an effluent compressor stream comprising acrylonitrile;
a line configured to convey, at a second pressure, the effluent compressor stream comprising acrylonitrile from the effluent compressor; and
an absorber configured to receive the effluent compressor stream comprising acrylonitrile from the line and allow for absorbing of the acrylonitrile in a second aqueous stream to provide a rich water comprising acrylonitrile.

17. The ammoxidation apparatus of claim 16, wherein the effluent compressor is configured to compress the quenched stream to provide an effluent compressor stream comprising acrylonitrile having pressure equal to the second pressure.

18. The apparatus of claim 16, wherein the second pressure (absolute) is about 2 to about 12 times greater than the first pressure.

19. The apparatus of claim 16, wherein the second pressure is about 300 kPa (absolute) to about 500 kPa (absolute).

20. The apparatus of claim 16, wherein the second aqueous stream has a temperature of about 4° C. to about 45° C.

21. The apparatus of claim 20, wherein the second aqueous stream has a temperature of about 20° C. to about 45° C.

22. The apparatus of claim 21, wherein the second aqueous stream has a flow rate of about 15 to about 20 kg/kg acrylonitrile produced.

23. The apparatus of claim 11, further comprising an expander configured to expand non-absorbed effluent from the absorber to reduce a pressure of the non-absorbed effluent.

24. The apparatus of claim 23, wherein the expander is configured to reduce the pressure of the non-absorbed effluent from the absorber to a pressure of about 150 kPa (absolute) or less.

25. The apparatus of claim 23, further comprising a pre-heater configured to pre-heat the non-absorbed effluent from the absorber prior being expanded in the expander.

26. The apparatus of claim 25, wherein the pre-heater is configured to raise the temperature of the non-absorbed effluent from a temperature of about 25° C. to about 40° C. to a temperature of about 350° C. or more.

27. The apparatus of claim 23, wherein the expander is configured to lower the temperature of the non-absorbed effluent from a temperature of about 300° C. to about 400° C. to a temperature in the range of about 200° C. to about 260° C.

28. The apparatus of claim 14, wherein the reactor has a linear velocity of about 0.5 to about 1.2 meters/second.

29. The apparatus of claim 14, wherein the reactor has an internal diameter of about 5 to about 15 meters.

30. The apparatus of claim 14, wherein the reactor has a height (tangent to tangent) of about 10 to about 25 meters.
